(19) United States
(12) Patent Application Publication
(10) Pub. No.: US 2026/0090820 A1
(43) Pub. Date: Apr. 2, 2026

(54) SURGICAL INSTRUMENT WITH MECHANICAL HARMONIC GENERATOR

(71) Applicant: CILAG GMBH INTERNATIONAL, ZUG (CH)

(72) Inventors: Brian W. Bear, Cincinnati, OH (US); Joshua D. Wilson, Cincinnati, OH (US)

(21) Appl. No.: 18/901,886

(22) Filed: Sep. 30, 2024

(51) Int. Cl. A61B 17/32; A61B 17/00

(52) U.S. Cl. CPC A61B 17/320092; A61B 2017/00137; A61B 2017/00398; A61B 2017/320075; A61B 2017/320094

(57) ABSTRACT

A surgical instrument includes an ultrasonic blade and a mechanical harmonic generator coupled to the ultrasonic blade. The mechanical harmonic generator includes an exciter wheel operatively coupled to an electric motor. The exciter wheel, when rotated by the electric motor, causes the ultrasonic blade to vibrate at a reference frequency. In some embodiments, the exciter wheel may be in contact with the ultrasonic blade and rotate on the ultrasonic blade by stick-slip motion. Additionally, the mechanical harmonic generator may include a resonator assembly and a tensile vibrator, and the exciter wheel may be contact with the resonator assembly or the tensile vibrator.

### TECHNICAL FIELD

The present disclosure relates generally to surgical instruments and, more particularly, to surgical instruments including an ultrasonic blade.

### BACKGROUND

Ultrasonic surgical instruments are finding increasingly widespread applications in surgical procedures by virtue of their unique performance characteristics. Depending upon specific device configurations and operational parameters, ultrasonic surgical instruments can provide contemporaneous transection of tissue and homeostasis by coagulation, which may reduce or otherwise minimize patient trauma. A typical ultrasonic surgical instrument may include a handpiece containing an ultrasonic transducer and an elongated shaft assembly having a distally mounted end effector (e.g., a jaw assembly including an ultrasonic blade and a clamp arm, where the clamp arm may comprise a non-stick tissue pad) to cut and seal tissue. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. The end effector transmits ultrasonic energy to tissue brought into contact with the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. Generally, a surgeon can control the cutting speed and coagulation by the force applied to the tissue by the end effector, the time over which the force is applied, and the selected excursion level of the end effector.

Typical ultrasonic surgical instruments include a generator, which may be separate from the instrument itself. The generator includes an electronic circuit configured to electrically generate an ultrasonic vibration of a blade of the surgical instrument at a desired frequency using a piezoelectric system (e.g., a stack of piezoelectric chips). However, such electronic generators can be cumbersome, costly, and complex, which may make such typical ultrasonic surgical instruments less practical for use in regions in which funding, electricity, and/or other resources are limited.

### SUMMARY

According to an aspect of the present disclosure, a surgical instrument may include and end effector and a mechanical harmonic generator. The end effector may include an ultrasonic blade. The mechanical harmonic generator may be coupled to the ultrasonic blade, and may include an exciter wheel operatively coupled to an electric motor. The exciter wheel, when rotated by the electric motor, may cause the ultrasonic blade to vibrate at a reference frequency.

In some embodiments, the end effector may include a jaw assembly movable between an open state and a closed state. Additionally, in some embodiments, the exciter wheel may be in contact with the ultrasonic blade and rotates on the ultrasonic blade by stick-slip motion.

In some embodiments, the mechanical harmonic generator may further include a force adjuster configured to selectively adjust a force between the exciter wheel and the ultrasonic blade. In such embodiments, to adjust the force between the exciter wheel and the ultrasonic blade may include to adjust a positon of the exciter wheel relative to the ultrasonic blade. In some embodiments, the surgical instrument may also include a battery configured to power the electric motor.

Additionally, in some embodiments, the mechanical harmonic generator may include a resonator assembly coupled to the ultrasonic blade. In such embodiments, the exciter wheel may be in contact with the resonator assembly and may rotate on the resonator assembly by stick-slip motion. Additionally, in such embodiments, rotation of the exciter wheel on the resonator assembly by the stick-slip motion may cause the resonator assembly to vibrate at the reference frequency. The resonator assembly, when vibrated by the exciter wheel, may cause the ultrasonic blade to vibrate at the reference frequency. In such embodiments, the reference frequency may be dependent on a size of the resonator assembly.

In some embodiments, the mechanical harmonic generator may include a resonator assembly coupled to the ultrasonic blade and a tensile vibrator coupled to the resonator assembly. The tensile vibrator may be held in tension by a mechanical ground. Additionally, the exciter wheel may be in contact with the tensile vibrator and rotate on the resonator assembly by stick-slip motion. In such embodiments, the rotation of the exciter wheel on the tensile vibrator by the stick-slip motion may cause the tensile vibrator to vibrate at the reference frequency. The tensile vibrator, when vibrated by the exciter wheel, may cause the resonator assembly to vibrate at the reference frequency. Additionally, the resonator assembly, when vibrated by the tensile vibrator, causes the ultrasonic blade to vibrate at the reference frequency. In some embodiments, the reference frequency may be dependent on an amount of tension applied to the tensile vibrator.

According to another aspect of the present disclosure, a surgical instrument may include an ultrasonic blade, an electric motor having a motor shaft, and an exciter wheel operatively coupled to the motor shaft of the electric motor and in contact with the ultrasonic blade. The electric motor may be configured to rotate the exciter wheel on the ultrasonic blade to cause the ultrasonic blade to vibrate at a reference frequency.

In some embodiments, the electric motor causes the exciter wheel to rotate on the ultrasonic blade by stick-slip motion. In some embodiments, the surgical instrument may further include a force adjuster configured to selectively adjust a force between the exciter wheel and the ultrasonic blade. Additionally, in some embodiments, to adjust the force between the exciter wheel and the ultrasonic blade may include to adjust a positon of the exciter wheel relative to the ultrasonic blade.

According to yet a further aspect of the present disclosure, a surgical instrument may include an ultrasonic blade, a resonator assembly coupled to the ultrasonic blade, an electric motor, and an exciter wheel operatively coupled to the electric motor and in contact with the ultrasonic blade. The exciter wheel, when rotated by the electric motor, may cause the resonator assembly to vibrate at a reference frequency. Additionally, the resonator assembly, when vibrated by the exciter wheel, may cause the ultrasonic blade to vibrate at the reference frequency.

In some embodiments, the exciter wheel may be in contact with the resonator assembly and may rotate on the resonator assembly by stick-slip motion. In such embodiments, rotation of the exciter wheel on the resonator assembly by the stick-slip motion may cause the resonator assembly to vibrate at the reference frequency.

Additionally, in some embodiments, the surgical instrument may further include a tensile vibrator coupled to the resonator assembly. In such embodiments, the exciter wheel may be in contact with the tensile vibrator and may rotate on the resonator assembly by stick-slip motion. Additionally, in such embodiments, rotation of the exciter wheel on the tensile vibrator by the stick-slip motion may cause the tensile vibrator to vibrate at the reference frequency. Additionally, the tensile vibrator, when vibrated by the exciter wheel, may cause the resonator assembly to vibrate at the reference frequency.

### BRIEF DESCRIPTION OF THE DRAWINGS

The detailed description particularly refers to the following figures, in which:

FIG. 1 is a side elevation view an embodiment of a surgical instrument including an ultrasonic blade and a mechanical harmonic generator configured to cause vibrations in the ultrasonic blade;

FIG. 2 is a perspective view of the surgical instrument of FIG. 1 including a remote power source;

FIG. 3 is a side elevation view of a jaw assembly of an end effector of the surgical instrument of FIG. 1 in an open state;

FIG. 4 is a side elevation view of the jaw assembly of the end effector of the surgical instrument of FIG. 1 in a closed state;

FIG. 5 is a simplified block diagram of an embodiment of a component assembly of the surgical instrument of FIG. 1;

FIG. 6 is a simplified block diagram of an embodiment of the mechanical harmonic generator of the surgical instrument of FIGS. 1 and 5;

FIG. 7 is a simplified illustration of an embodiment of the mechanical harmonic generator of FIG. 6;

FIG. 8 is a simplified illustration of another embodiment of the mechanical harmonic generator of FIG. 6;

FIG. 9 is a simplified illustration of a further embodiment of the mechanical harmonic generator of FIG. 6;

FIG. 10 is a simplified illustration of an additional embodiment of the mechanical harmonic generator of FIG. 6;

FIGS. 11 and 12 is a simplified illustration of an exciter wheel of the mechanical harmonic generator of FIG. 10 rotating on the ultrasonic blade by slip-stick motion; and

FIG. 13 is a simplified graph of a force generated between the exciter wheel of the mechanical harmonic generator of FIG. 10 and the ultrasonic blade during the slip-stick motion of the exciter wheel on the ultrasonic blade; and

FIG. 14 is a simplified illustration of a wheel force adjuster of the mechanical harmonic generator of FIG. 6.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

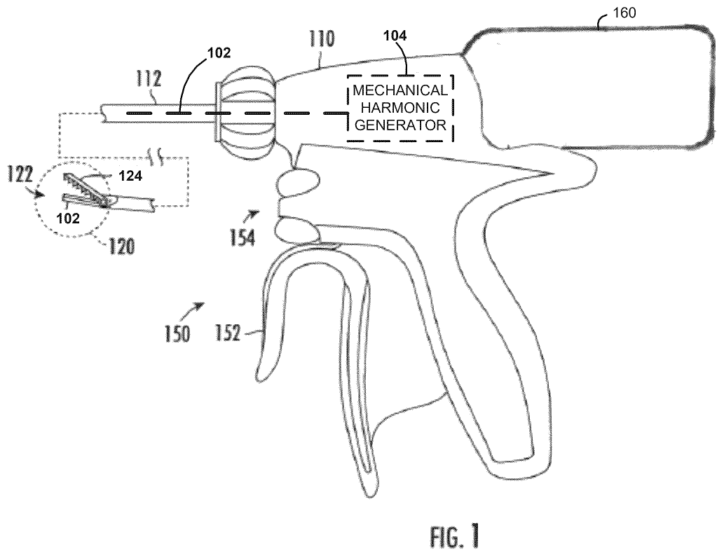

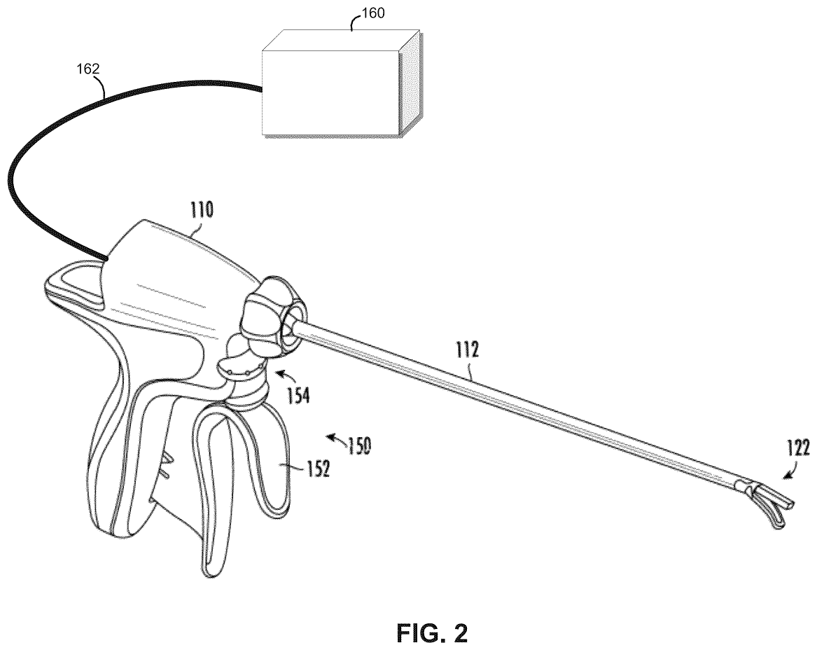

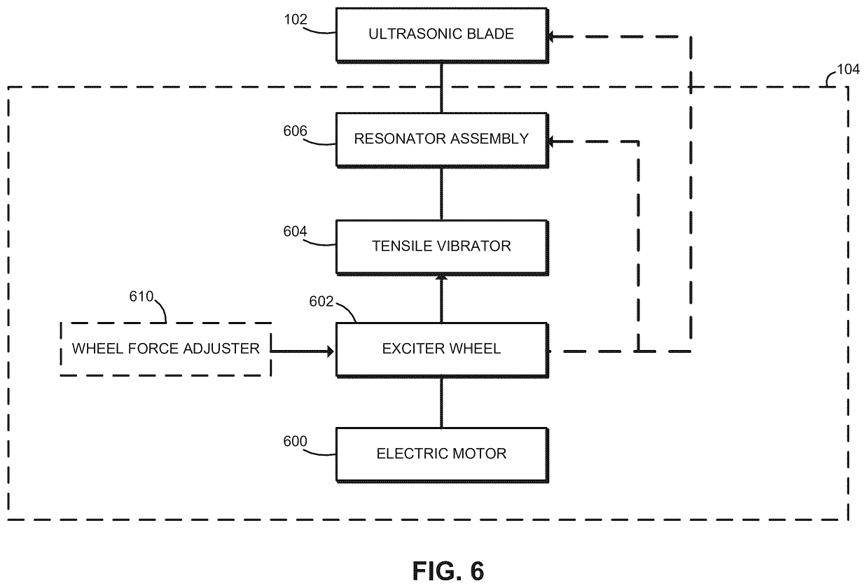

Referring now to FIG. 1, in an illustrative embodiment, an ultrasonic surgical instrument 100 includes an ultrasonic blade 102 and a mechanical harmonic generator 104. The ultrasonic surgical instrument 100 is usable by a surgeon to perform various surgical procedures including laparoscopic, endoscopic, or traditional open surgical procedures. In doing so, the surgeon may selectively activate an ultrasonic mode of the ultrasonic surgical instrument 100. In the ultrasonic mode, the mechanical harmonic generator 104 is configured to cause the ultrasonic blade 102 to vibrate at a reference frequency by mechanical mechanisms (i.e., not by the use of the piezoelectric effect), which facilitates the contemporaneous cutting and hemostatic sealing of patient tissue. For example, as discussed in more detail below, the illustrative mechanical harmonic generator 104 includes an electric motor 600 and an exciter wheel 602 (see FIG. 6), that is driven by the electric motor 600. The exciter wheel 602 contacts the ultrasonic blade 102 or an intervening harmonic component and generates vibration motion in the ultrasonic blade 102 (either directly or through the one or more harmonic components) via stick-slip motion (i.e., motion exhibiting the stick-slip phenomenon), similar to the movement of a bow across a music string. By using only the electric motor 600 and the exciter wheel 602 (and other harmonic components, in some embodiments) to generate the ultrasonic vibration of the blade 102, the overall complexity and cost of the ultrasonic surgical instrument 100 can be reduced relative to ultrasonic surgical instruments utilizing electronic generators to produce such ultrasonic vibrations.

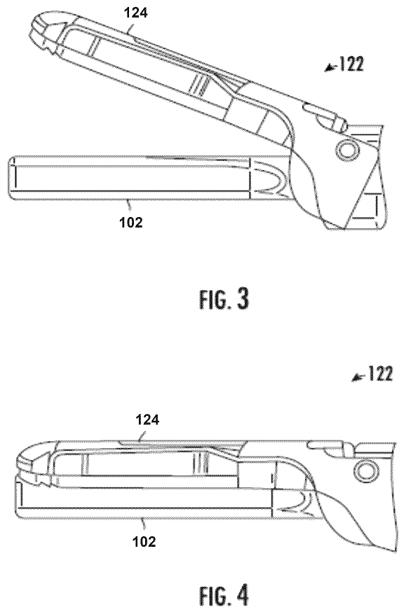

The ultrasonic surgical instrument 100 is illustratively embodied as surgical shears but may be embodied as other types of surgical instruments having an ultrasonic mode in other embodiments. In the illustrative embodiment, the ultrasonic surgical instrument also includes a handle assembly and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 illustratively includes a jaw assembly 122, which includes the ultrasonic blade 102 and a corresponding jaw clamp 124. As shown in FIGS. 3 and 4, the illustrative jaw assembly 122 is movable between an open state (FIG. 3) in which the jaw clamp 124 is positioned away from the ultrasonic blade 102 and a closed state (FIG. 4) in which the jaw clamp 124 is positioned near or otherwise contacts the ultrasonic blade 102. Actuation of the jaw assembly 122 from the open state to the closed state allows for the grasping, cutting, and coagulation of vessels and/or tissue by the jaw assembly 122. However, in embodiments in which the ultrasonic surgical instrument 100 is not embodied as surgical shears, the end effector 120 may not include a jaw assembly 122. In such embodiments, the end effector 120 may include only the ultrasonic blade 102 (i.e., the surgical instrument may be embodied as a surgical knife or blade, rather than shears) or other components, depending on the type of surgical instrument.

Referring back to FIG. 1, the handle assembly 110 includes a trigger assembly 150, which includes a primary trigger 152 and a switch assembly 154. In the illustrative embodiment, the primary trigger 152 is operable by the surgeon to move the jaw assembly 122 of the end effector 120 between the open and closed states. The switch assembly 154 includes one or more buttons, which are selectable by the surgeon to activate the ultrasonic mode of the ultrasonic surgical instrument 100. That is, the a surgeon may select one of the buttons of the switch assembly 154 to activate the electric motor 600 of the mechanical harmonic generator 104 to cause rotation of the exciter wheel 602. As discussed in more detail below, the rotation of the exciter wheel 602 results in stick-slip motion of the exciter wheel 602 on the ultrasonic blade 102 itself or on other harmonic components, which causes the ultrasonic blade 102 to vibrate at a reference or resonate frequency. For example, the stick-slip motion of the exciter wheel 602 may cause the ultrasonic blade 102 to vibrate longitudinally in the range of, for example, approximately 20 kHz to 250 kHz. In particular embodiments, for example, the ultrasonic blade 102 may vibrate in the range of about 54 kHz to 56 kHz (e.g., at about 55.5 kHz). In other embodiments, the ultrasonic blade 102 may vibrate at other frequencies including, for example, about 31 kHz or about 80 kHz. In some embodiments, the frequency of the vibrations at the ultrasonic blade 102 can be controlled by, for example, the force exerted by the exciter wheel 602, the speed of the electric motor 600, and/or adjustments to the other harmonic components as discussed in more detail below.

In the illustrative embodiment, the ultrasonic surgical instrument 100 includes a battery power source 160, which may be received in the handle assembly 110 as shown in FIG. 1. The battery power source 160 is configured to provide power to the electric motor 600 and other components of the ultrasonic surgical instrument 100. The battery power source 160 may be replaceable and/or rechargeable, allowing the ultrasonic surgical instrument 100 to be used in remote locations that may not have reliable energy sources. As shown in FIG. 1, in some embodiments, the battery power source 160 may be remote from the handle assembly 110 but connected thereto via a power cable 162.

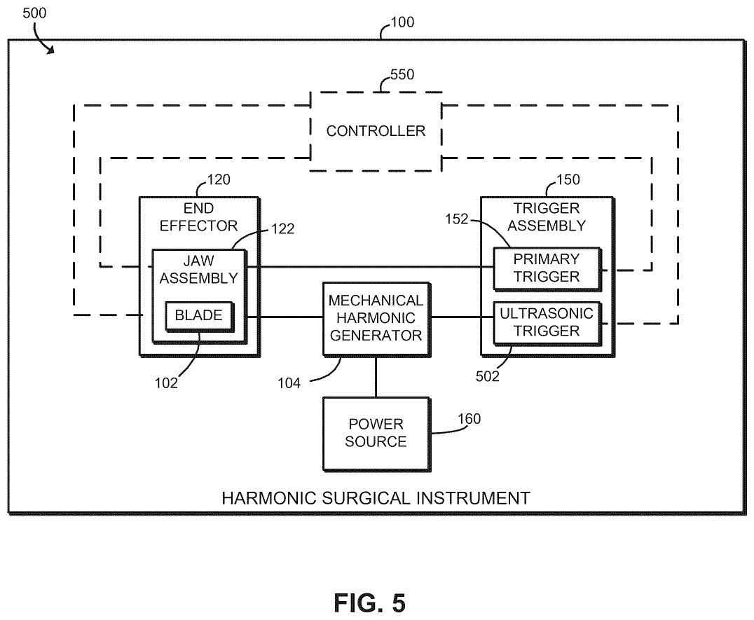

Referring now to FIG. 5, the ultrasonic surgical instrument includes a component assembly 500. The component assembly 500 includes the mechanical harmonic generator 104, the end effector 120, the trigger assembly 150, and the battery power source 160. The trigger assembly 150 includes the primary trigger 152, which is operably coupled to the jaw assembly and is selectable to actuate the jaw assembly 122. The trigger assembly 150 also includes an ultrasonic trigger 502, which is operably coupled to the mechanical harmonic generator 104 and is selectable to control operation of the electric motor 600. The mechanical harmonic generator 104 is coupled to the ultrasonic blade 102 of the end effector 120 and includes the electric motor 600 and the exciter wheel 602. The exciter wheel 602, when rotated by the electric motor 600, is configured to cause the ultrasonic blade 102 to vibrate at a reference frequency. The battery power source 160 is electrically connected to the mechanical harmonic generator 104 and provides power to the electric motor 600 as needed.

Although the illustrative ultrasonic surgical instrument 100 is designed for low complexity, the component assembly 500 of the ultrasonic surgical instrument 100 may include a controller 550 in some embodiments. In such embodiments, the controller 550 may be embodied as any type of controller, functional block, digital logic, or other component, device, circuitry, or collection thereof capable of performing the functions described herein. For example, the controller 550 may include a processor, memory, and an input/output (I/O) subsystem. The controller 550 may be configured to control certain operations of the ultrasonic surgical instrument 100. For example, in some embodiments, the controller 550 may control the activation and/or speed of the electric motor 600 of the mechanical harmonic generator 104. Additionally, in some embodiments, the jaw assembly 122 may be motorized and, in such embodiments, the controller 550 may control the motorized actuation of the jaw assembly 122.

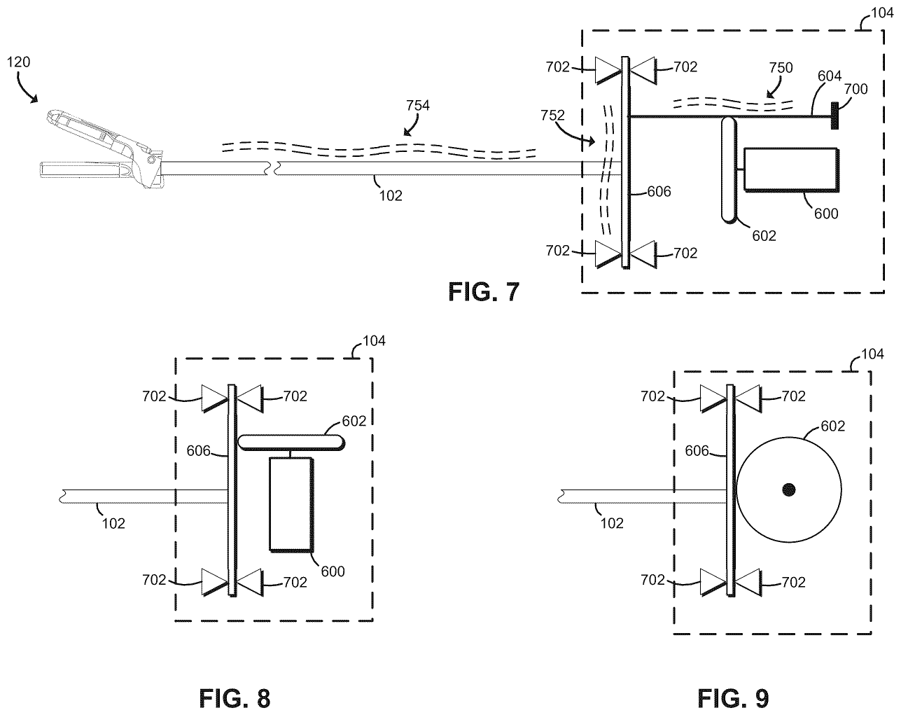

Referring now to FIG. 6, in an illustrative embodiment, the mechanical harmonic generator 104 includes the electric motor 600, the exciter wheel 602, a tensile vibrator 604, and a resonator assembly 606. In such embodiments, the exciter wheel 602 is in direct contact with the tensile vibrator 604 and is configured to rotate on the tensile vibrator 604 by stick-slip motion as discussed in more detail below in regard to FIG. 7.

The electric motor 600 may be embodied as type of electric motor cable of driving or rotating the exciter wheel 602. For example, in the illustrative embodiment, the electric motor 600 is embodied as a direct current (DC) motor configured to receive power from a DC power source 160. The exciter wheel 602 is sized and shaped so as to be able to rotate on the tensile vibrator 604 (or the resonator assembly 606 or ultrasonic blade 102, depending on the embodiment). The exciter wheel 602 may be made from any suitable material that allows for or otherwise promotes stick-slip motion of the exciter wheel 602 on the tensile vibrator 604, resonator assembly 606, or ultrasonic blade 102. For example, in the illustrative embodiment, the exciter wheel 602 is formed from a polymer such as rubber or plastic material. In some embodiments, the exciter wheel 602 may have a core formed from a different material (e.g., a metal core) and an outer cover formed from a material that promotes stick-slip motion (e.g., a polymer material). Additionally, in some embodiments, the exciter wheel 602 may have a coating on the outer surface that further promotes stick-slip motion. For example, the coating may be a rosin material, a granular material, or other material that modifies (e.g., increases) the friction of the exciter wheel 602 on the tensile vibrator 604, resonator assembly 606, or ultrasonic blade 102.

The tensile vibrator 604 may be formed from any material capable of being held in tension and vibrating at a reference frequency in response to excitation by the exciter wheel 602. For example, the tensile vibrator 604 may be formed from a metal or polymer material and have a decreased thickness relative to other components (e.g., the thickness of the tensile vibrator 604 may be less than the ultrasonic blade 102). The tensile vibrator 604 is physically coupled, attached, or secured to the resonator assembly 606.

Similarly, the resonator assembly 606 may be formed from any material capable of vibrating at a reference frequency in response to excitation by the tensile vibrator 604 (or directly by the exciter wheel 602, depending on the embodiment). For example, the resonator assembly 606 may be formed as a metal or polymer material and have a disc or otherwise relatively flat shape. Additionally, the resonator assembly 606 may have a decreased thickness relative to other components (e.g., the thickness of the resonator assembly 606 may be less than the ultrasonic blade 102). The resonator assembly 606 is coupled to the ultrasonic blade 102 in a manner through which vibrations of the resonator assembly 606 are transferred to the ultrasonic blade 102.

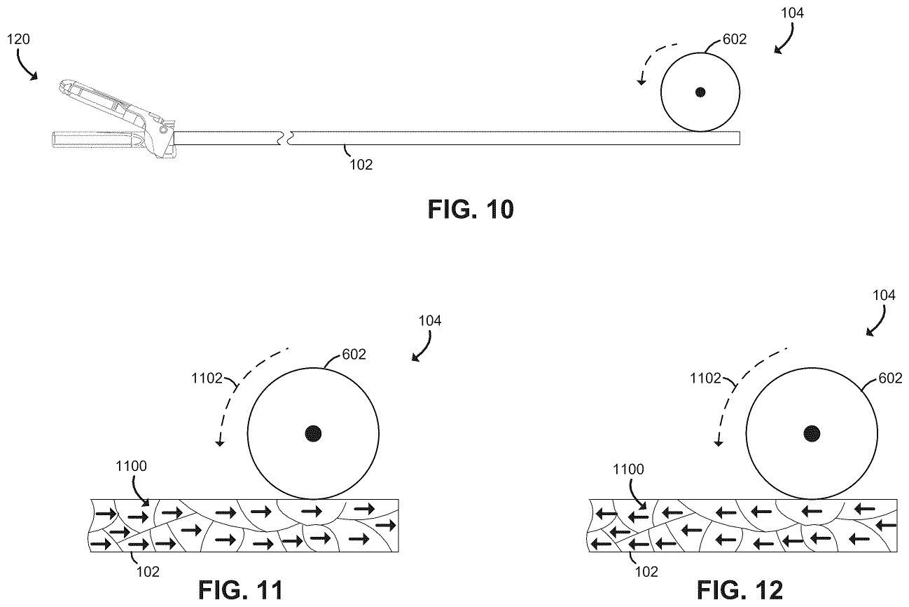

Although the illustrative embodiment of the mechanical harmonic generator 104 includes each of the tensile vibrator 604 and the resonator assembly 606, the mechanical harmonic generator 104 may not include the tensile vibrator 604 in other embodiments. In such embodiments, the exciter wheel 602 is in direct contact with the resonator assembly 606 as indicated by the dashed arrow of FIG. 6 and is configured to rotate on the resonator assembly 606 by stick-slip motion as discussed in more detail below in regard to FIGS. 8 and 9. Additionally, in yet other embodiments, the mechanical harmonic generator 104 may not include the tensile vibrator 604 or the resonator assembly 606. In such embodiments, the exciter wheel 602 is in direct contact with the ultrasonic blade 102 as indicated by the dashed arrow of FIG. 6 and is configured to rotate on the ultrasonic blade 102 by stick-slip motion as discussed in more detail below in regard to FIGS. 10-13.

In some embodiments, the mechanical harmonic generator 104 may also include a wheel force adjuster 610. The wheel force adjuster 610 is configured to adjust the force exerted by the exciter wheel 602 on the tensile vibrator 604, the resonator assembly 606, or the ultrasonic blade 102, depending on which component the exciter wheel 602 is in direct contact with. As discussed in more detail below in regard to FIG. 14, the wheel force adjuster 610 may be a manual, mechanical adjuster configured to adjust the relative positioning of the exciter wheel 602 to thereby modify the exerted force. In other embodiments, the wheel force adjuster 610 may include an electric motor that is operable to adjust the relative positing of the exciter wheel 602 (e.g., by adjusting the relative position of the electric motor 600).

Referring now to FIG. 7, an illustrative embodiment in which the mechanical harmonic generator 104 includes each of the tensile vibrator 604 and the resonator assembly 606 is shown. The electric motor 600 and the exciter wheel 602 are arranged such that the exciter wheel 602 is in direct contact with the tensile vibrator 604. The tensile vibrator 604 is coupled to the resonator assembly 606 on one end and held in tension by a mechanical ground 700 at an opposite end. The mechanical ground 700 may be embodied as any suitable structure capable of holding the tensile vibrator 604 at a degree of tension. The particular reference frequency at which tensile vibrator 604 vibrates may be dependent upon the degree at which the tensile vibrator 604 is held in tension by the mechanical ground 700.

Similar to the tensile vibrator 604, the resonator assembly 606 is secured by a set of mechanical supports 702, which may be embodied as any suitable structure capable of supporting the resonator assembly 606 while allowing the resonator assembly 606 to vibrate. The number and structure of the mechanical supports 702 may depend on the shape and size of the resonator assembly 606 and/or other parameters of the particular embodiment. The resonator assembly 606 is coupled to the tensile vibrator 604 as discussed above and to the ultrasonic blade 102. For example, the resonator assembly 606 may be attached or coupled to the ultrasonic blade 102 via connections configured to transfer vibrations of the resonator assembly 606 to the ultrasonic blade 102.

In use, the electric motor 600 rotates the exciter wheel 602, which is in contact with the tensile vibrator 604. Rotation of the exciter wheel 602 by the electric motor 600 causes the exciter wheel 602 to rotate on the tensile vibrator 604 by stick-slip motion, which causes the tensile vibrator 604 to vibrate at a reference frequency as indicated by vibration waves 750 in FIG. 7 (e.g., in the range of 20 kHz to 250 kHz, 54 kHz to 56 kHz, or about 55.5 kHz). The tensile vibrator 604, in response to being vibrated by the exciter wheel 602, causes the by resonator assembly 606 to vibrate at the reference frequency as indicated by the vibration waves 752 of FIG. 7. In some embodiments, the resonator assembly 606 may be configured to amplify the magnitude of the vibration provided by the tensile vibrator 604. Regardless, in response to being vibrated by the tensile vibrator 604, the resonator assembly 606 causes the ultrasonic blade 102 to vibrate at the reference frequency as indicated by the vibration waves 754 of FIG. 7. In this way, the mechanical harmonic generator 104 is configured to vibrate the ultrasonic blade 102 at a reference frequency by mechanical mechanisms without the use of the piezoelectric effect.

Referring now to FIGS. 8 and 9, in other embodiments as discussed above, the mechanical harmonic generator 104 may not include the tensile vibrator 604. In such embodiments, the electric motor 600 and the exciter wheel 602 are arranged such that the exciter wheel 602 is in direct contact with the resonator assembly 606. Again, the resonator assembly 606 is supported by the mechanical supports 702 and coupled to the ultrasonic blade 102 (e.g., via connections configured to transfer vibrations of the resonator assembly 606 to the ultrasonic blade 102). The electric motor 600 and the exciter wheel 602 may be arranged in any one of a number of orientations, relative to the resonator assembly 606, that allow the exciter wheel to contact the resonator assembly 606 as shown comparatively in FIGS. 8 and 9.

In use, the electric motor 600 rotates the exciter wheel 602, which is in contact with the resonator assembly 606. Rotation of the exciter wheel 602 by the electric motor 600 causes the exciter wheel 602 to rotate on the resonator assembly 606 by stick-slip motion, which causes the resonator assembly 606 to vibrate at a reference frequency. Again, in some embodiments, the resonator assembly 606 may be configured to amplify the magnitude of the vibration provided by the exciter wheel 602. Regardless, in response to being vibrated by the exciter wheel 602, the resonator assembly 606 causes the ultrasonic blade 102 to vibrate at the reference frequency. In this way, the mechanical harmonic generator 104 is configured to vibrate the ultrasonic blade 102 at a reference frequency by mechanical mechanisms without the use of the piezoelectric effect.

Referring now to FIGS. 10-13, in other embodiments as discussed above, the mechanical harmonic generator 104 may not include either tensile vibrator 604 or the resonator assembly 606. In such embodiments, the electric motor 600 and the exciter wheel 602 are arranged such that the exciter wheel 602 is in direct contact with the ultrasonic blade 102 as shown in FIG. 10. In use, rotation of the exciter wheel 602 by the electric motor 600 causes the exciter wheel 602 to rotate on the ultrasonic blade 102 by stick-slip motion, which causes the ultrasonic blade 102 to vibrate at a reference frequency. As shown in FIGS. 11 and 12, the stick-slip motion of the exciter wheel 602 on the ultrasonic blade 102 is caused by friction between the exciter wheel 602 and the ultrasonic blade 102. The friction causes different portions 1100 of the ultrasonic blade 102 to “stretch” in the direction 1102 of the rotation of the exciter wheel 602. At some point in time, however, the force of the stretching ultrasonic blade 102 overcomes the frictional force between the exciter wheel 602 and the ultrasonic blade 102. At such point in time, the ultrasonic blade 102 slips back to its original equilibrium as shown in FIG. 12.

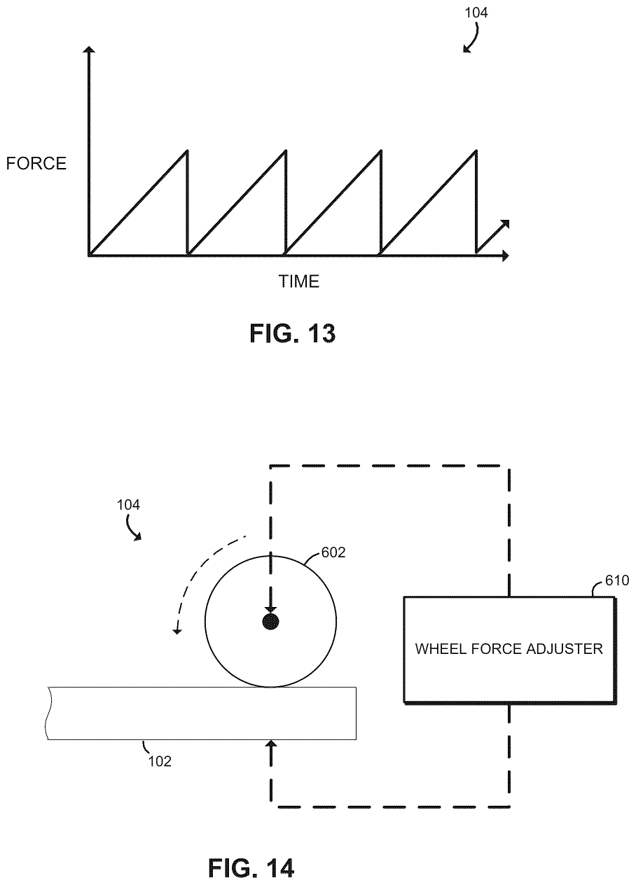

The “stretching” and slipping of the ultrasonic blade 102 due to the stick-slip motion of the exciter wheel 602 causes an oscillating vibration as shown in graph 1300 of FIG. 13. Because the resulting oscillation vibration has sharp impulses (similar to a struck tuning fork), the oscillation vibration causes the ultrasonic blade to resonate at its natural resonance frequency. It should be appreciated that a similar oscillation vibration is caused in the tensile vibrator 604 and in the resonator assembly 606 in those embodiments in which the exciter wheel 602 is in direct contact with those components.

Referring now to FIG. 14, as discussed above, the mechanical harmonic generator 104 may include a wheel force adjuster 610 in some embodiments. The wheel force adjuster 610 is operable, either manually or via a motor, to adjust the force of the exciter wheel 602 on the ultrasonic blade 102 (or on the tensile vibrator 604 or the resonator assembly 606, depending on the embodiment). To do so, the wheel force adjuster 610 may adjust the relative position of the exciter wheel 602 and electric motor 600 to thereby increase or decrease the force exerted by the exciter wheel 602. It should be appreciated that as the force exerted by the exciter wheel 602 increases, the friction between the exciter wheel 602 and the ultrasonic blade 102/tensile vibrator 604/resonator assembly 606 likewise increases. The increase in friction of the exciter wheel 602 causes the ultrasonic blade 102/tensile vibrator 604/resonator assembly 606 to stretch further, thereby increasing the energy supplied into the system.

As such, some embodiments of the mechanical harmonic generator 104 may include various mechanisms for adjusting the position of the exciter wheel 602 relative to the ultrasonic blade 102 or other components of the mechanical harmonic generator 104 as described above. The resulting force exerted by the exciter wheel 602, or related adjustments, may serve to control the operating conditions of the ultrasonic blade and/or mechanical harmonic generator 104 to “optimize” the vibration frequency of and the power delivered to the ultrasonic blade 102. Such adjustments in the force exerted by the exciter wheel 602 may be automated or under the control of surgeon (i.e., the user of the ultrasonic surgical instrument 100).

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A surgical instrument comprising:
an end effector having an ultrasonic blade; and
a mechanical harmonic generator coupled to the ultrasonic blade, wherein the mechanical harmonic generator includes an exciter wheel operatively coupled to an electric motor, wherein the exciter wheel, when rotated by the electric motor, causes the ultrasonic blade to vibrate at a reference frequency.

2. The surgical instrument of claim 1, wherein the end effector includes a jaw assembly movable between an open state and a closed state.

3. The surgical instrument of claim 1, wherein the exciter wheel is in contact with the ultrasonic blade and rotates on the ultrasonic blade by stick-slip motion.

4. The surgical instrument of claim 1, wherein the mechanical harmonic generator further includes a force adjuster configured to selectively adjust a force between the exciter wheel and the ultrasonic blade.

5. The surgical instrument of claim 4, wherein to adjust the force between the exciter wheel and the ultrasonic blade comprises to adjust a positon of the exciter wheel relative to the ultrasonic blade.

6. The surgical instrument of claim 1, wherein the mechanical harmonic generator includes a resonator assembly coupled to the ultrasonic blade, and wherein the exciter wheel is in contact with the resonator assembly and rotates on the resonator assembly by stick-slip motion.

7. The surgical instrument of claim 6, wherein rotation of the exciter wheel on the resonator assembly by the stick-slip motion causes the resonator assembly to vibrate at the reference frequency, and wherein the resonator assembly, when vibrated by the exciter wheel, causes the ultrasonic blade to vibrate at the reference frequency.

8. The surgical instrument of claim 7, wherein the reference frequency is dependent on a size of the resonator assembly.

9. The surgical instrument of claim 1, wherein the mechanical harmonic generator includes a resonator assembly coupled to the ultrasonic blade and a tensile vibrator coupled to the resonator assembly, wherein the tensile vibrator is held in tension by a mechanical ground, and
wherein the exciter wheel is in contact with the tensile vibrator and rotates on the tensile vibrator by stick-slip motion.

10. The surgical instrument of claim 9, wherein rotation of the exciter wheel on the tensile vibrator by the stick-slip motion causes the tensile vibrator to vibrate at the reference frequency, and wherein the tensile vibrator, when vibrated by the exciter wheel, causes the resonator assembly to vibrate at the reference frequency, and wherein, the resonator assembly, when vibrated by the tensile vibrator, causes the ultrasonic blade to vibrate at the reference frequency.

11. The surgical instrument of claim 10, wherein the reference frequency is dependent on an amount of tension applied to the tensile vibrator.

12. The surgical instrument of claim 1, further comprising a battery configured to power the electric motor.

13. A surgical instrument comprising:
an ultrasonic blade;
an electric motor having a motor shaft;
an exciter wheel operatively coupled to the motor shaft of the electric motor and in contact with the ultrasonic blade, and
wherein the electric motor is configured to rotate the exciter wheel on the ultrasonic blade to cause the ultrasonic blade to vibrate at a reference frequency.

14. The surgical instrument of claim 13, wherein the electric motor causes the exciter wheel to rotate on the ultrasonic blade by stick-slip motion.

15. The surgical instrument of claim 13, further comprising a force adjuster configured to selectively adjust a force between the exciter wheel and the ultrasonic blade.

16. The surgical instrument of claim 15, wherein to adjust the force between the exciter wheel and the ultrasonic blade comprises to adjust a positon of the exciter wheel relative to the ultrasonic blade.

17. A surgical instrument comprising:
an ultrasonic blade;
a resonator assembly coupled to the ultrasonic blade
an electric motor;
an exciter wheel operatively coupled to the electric motor and in contact with the ultrasonic blade, and
wherein the exciter wheel, when rotated by the electric motor, causes the resonator assembly to vibrate at a reference frequency, and wherein the resonator assembly, when vibrated by the exciter wheel, causes the ultrasonic blade to vibrate at the reference frequency.

18. The surgical instrument of claim 17, wherein the exciter wheel is in contact with the resonator assembly and rotates on the resonator assembly by stick-slip motion, and wherein rotation of the exciter wheel on the resonator assembly by the stick-slip motion causes the resonator assembly to vibrate at the reference frequency.

19. The surgical instrument of claim 17, further comprising a tensile vibrator coupled to the resonator assembly, wherein the exciter wheel is in contact with the tensile vibrator and rotates on the resonator assembly by stick-slip motion.

20. The surgical instrument of claim 19, wherein rotation of the exciter wheel on the tensile vibrator by the stick-slip motion causes the tensile vibrator to vibrate at the reference frequency, and wherein the tensile vibrator, when vibrated by the exciter wheel, causes the resonator assembly to vibrate at the reference frequency.
